(19) United States
(12) Patent Application Publication
(10) Pub. No.: US 2025/0352368 A1
(43) Pub. Date: Nov. 20, 2025

(54) STENT DESIGN FOR ENHANCED DRAINAGE

(71) Applicant: BOSTON SCIENTIFIC SCIMED, INC., Maple Grove, MN (US)

(72) Inventor: MARTYN G. FOLAN, Galway (IE)

(73) Assignee: BOSTON SCIENTIFIC SCIMED, INC., Maple Grove, MN (US)

(21) Appl. No.: 19/210,685

(22) Filed: May 16, 2025

Related U.S. Application Data
(60) Provisional application No. 63/649,166, filed on May 17, 2024.

(51) Int. Cl. A61F 2/88; A61F 2/94

(52) U.S. Cl. CPC A61F 2/88; A61F 2/94; A61F 2220/0016

(57) ABSTRACT

A polymeric stent is adapted for placement within a body cavity to provide a primary drainage passageway and a secondary drainage passageway for the body cavity. The polymeric stent includes a polymeric tubular body including a first end region, a second end region and an intervening intermediate region. The polymeric tubular body has a remembered configuration in which the intervening intermediate region of the polymeric tubular body forms a plurality of tightly arranged helical coil windings that define the primary drainage passageway and a delivery configuration different from the remembered configuration when constrained by a delivery device.

### CROSS-REFERENCE TO RELATED APPLICATIONS

The application claims the benefit of U.S. Provisional Patent Application Ser. No. 63/649,166, filed on May 17, 2024, the disclosure of which is incorporated herein by reference.

### TECHNICAL FIELD

The present disclosure relates generally to methods and apparatuses for various digestive ailments. More particularly, the disclosure relates to different configurations and methods of manufacture and use of a stent.

### BACKGROUND

Implantable stents are devices that are placed in a body structure, such as a blood vessel, esophagus, trachea, biliary tract, colon, intestine, stomach or body cavity, to provide support and to maintain the structure open. These devices are manufactured by any one of a variety of different manufacturing methods and may be used according to any one of a variety of methods. Of the known medical devices, delivery systems, and methods, each has certain advantages and disadvantages. There is an ongoing need to provide alternative medical devices and delivery devices as well as alternative methods for manufacturing and using medical devices and delivery devices.

### SUMMARY

The disclosure is directed to several alternative designs, materials and methods of manufacturing medical device structures and assemblies, and the use thereof.

An example may be found in a polymeric stent adapted for placement within a body cavity to provide a primary drainage pathway and a secondary drainage pathway for the body cavity. The polymeric stent includes a polymeric tubular body including a first end region, a second end region and an intervening intermediate region. The polymeric tubular body has a remembered configuration in which the intervening intermediate region of the polymeric tubular body forms a plurality of tightly arranged helical coil windings that together define the primary drainage pathway. The polymeric tubular body has a delivery configuration different from the remembered configuration when constrained by a delivery device.

Alternatively or additionally, the polymeric tubular body may define a lumen extending therethrough that defines the secondary drainage pathway.

Alternatively or additionally, the polymeric tubular body may further include one or more drainage ports fluidly coupled with the lumen.

Alternatively or additionally, the polymeric tubular body may include a shape memory polymer.

Alternatively or additionally, the polymeric stent may further include a shape memory element secured relative to the polymeric tubular body.

Alternatively or additionally, the polymeric stent may further include one or more anti-migration features disposed within at least one of the first end region and the second end region.

Alternatively or additionally, the plurality of tightly arranged helical coil windings defining the primary drainage pathway may be substantially orthogonal to a longitudinal axis of the polymeric stent.

Another example may be found in a polymeric stent adapted for placement within a body cavity to provide drainage through the body cavity. The polymeric stent includes a shape memory polymer tubular body defining a lumen extending therethrough, the shape memory polymeric tubular body having a delivery configuration when constrained by a delivery device and a deployed configuration when not constrained by a delivery device. When in the deployed configuration, the shape memory polymeric tubular body forms a plurality of tightly arranged helical coil windings that together define a drainage pathway.

Alternatively or additionally, the shape memory polymeric tubular body may include a first end region, a second end region and an intervening intermediate region that forms the plurality of tightly arranged helical coil windings defining the primary drainage passageway.

Alternatively or additionally, at least one of the first end region and the second end region may include one or more anti-migration features.

Alternatively or additionally, the polymeric stent may further include one or more drainage ports disposed within the polymeric tubular body and fluidly coupled with the lumen.

Alternatively or additionally, the polymeric stent may further include a shape memory element secured relative to the polymeric tubular body.

Another example may be found in a polymeric stent adapted for placement within a body cavity to provide a primary drainage pathway and a secondary drainage pathway for the body cavity. The polymeric stent includes a polymeric tubular body including a first end region, a second end region and an intervening intermediate region, and one or more anti-migration features disposed within at least one of the first end region and the second end region. The polymeric tubular body has a remembered configuration in which the intervening intermediate region of the polymeric tubular body forms a plurality of tightly arranged helical coil windings that together define the primary drainage pathway and has a delivery configuration different from the remembered configuration when constrained by a delivery device.

Alternatively or additionally, the one or more anti-migration features may include a barb.

Alternatively or additionally, the one or more anti-migration features may include a portion of the polymeric tubular body coiled into a coil winding.

Another example may be found in a polymeric stent adapted for placement within a body cavity to provide a primary drainage pathway and a secondary drainage pathway for the body cavity. The polymeric stent includes a polymeric tubular body including a first end region, a second end region and an intervening intermediate region. The polymeric tubular body has a remembered configuration in which the intervening intermediate region of the polymeric tubular body forms a plurality of tightly arranged helical coil windings that together define the primary drainage pathway. The polymeric tubular body has a delivery configuration different from the remembered configuration when constrained by a delivery device.

Alternatively or additionally, the polymeric tubular body may define a lumen extending therethrough that defines the secondary drainage pathway.

Alternatively or additionally, the polymeric tubular body may further include one or more drainage ports fluidly coupled with the lumen.

Alternatively or additionally, the polymeric tubular body may include a shape memory polymer.

Alternatively or additionally, the polymeric stent may further include a shape memory element secured relative to the polymeric tubular body.

Alternatively or additionally, the polymeric stent may further include one or more anti-migration features disposed within at least one of the first end region and the second end region.

Alternatively or additionally, the one or more anti-migration features may include a barb.

Alternatively or additionally, the one or more anti-migration features may include a portion of the polymeric tubular body coiled into a coil winding.

Alternatively or additionally, the plurality of tightly arranged helical coil windings defining the primary drainage path may be substantially orthogonal to a longitudinal axis of the polymeric stent.

Another example may be found in a polymeric stent adapted for placement within a body cavity to provide drainage through the body cavity. The polymeric stent includes a shape memory polymer tubular body defining a lumen extending therethrough, the shape memory polymeric tubular body having a delivery configuration when constrained by a delivery device and a deployed configuration when not constrained by a delivery device. When in the deployed configuration, the shape memory polymeric tubular body forms a plurality of tightly arranged helical coil windings that together define a drainage pathway.

Alternatively or additionally, the shape memory polymeric tubular body may include a first end region, a second end region and an intervening intermediate region that forms the plurality of tightly arranged helical coil windings defining the primary drainage pathway.

Alternatively or additionally, at least one of the first end region and the second end region may include one or more anti-migration features.

Alternatively or additionally, the one or more anti-migration features may include a barb.

Alternatively or additionally, the one or more anti-migration features may include a portion of the polymeric tubular body coiled into a coil winding.

Alternatively or additionally, the polymeric stent may further include one or more drainage ports disposed within the polymeric tubular body and fluidly coupled with the lumen.

Alternatively or additionally, the polymeric stent may further include a shape memory element secured relative to the polymeric tubular body.

Another example may be found in a polymeric stent adapted for placement within a body cavity to provide a primary drainage pathway and a secondary drainage pathway for the body cavity. The the polymeric stent includes a polymeric tubular body including a first end region, a second end region and an intervening intermediate region and one or more anti-migration features disposed within at least one of the first end region and the second end region. The polymeric tubular body has a remembered configuration in which the intervening intermediate region of the polymeric tubular body forms a plurality of tightly arranged helical coil windings that together define the primary drainage pathway and a delivery configuration different from the remembered configuration when constrained by a delivery device.

Alternatively or additionally, the one or more anti-migration features may include a barb.

Alternatively or additionally, the one or more anti-migration features may include a portion of the polymeric tubular body coiled into a coil winding.

Alternatively or additionally, the polymeric tubular body may include a lumen extending therethrough, the lumen defining the secondary drainage pathway.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic view of an illustrative polymeric stent shown in a delivery configuration;

FIG. 2 is a schematic view of the illustrative polymeric stent of FIG. 1, shown in an intermediate configuration;

FIG. 3 is a schematic view of the illustrative polymeric stent of FIG. 1, shown in a deployed configuration;

FIG. 4 is a schematic end view of the illustrative polymeric stent of FIG. 1, shown in a deployed configuration;

FIGS. 5 through 7 are schematic views of the illustrative polymeric stent of FIG. 1, showing a step by step process for deploying within a body cavity;

FIG. 8 is a schematic view of an illustrative polymeric stent deployed within a body cavity, showing a first level of coil expansion;

FIG. 9 is a schematic view comparing relative diameters of a primary drainage path and a secondary drainage path, showing how the level of coil expansion in FIG. 8 impacts an effective diameter of the primary drainage path;

FIG. 10 is a schematic view of an illustrative polymeric stent deployed within a body cavity, showing a second level of coil expansion greater than the first level of coil expansion illustrated in FIG. 8;

FIG. 11 is a schematic view comparing relative diameters of a primary drainage path and a secondary drainage path, showing how the level of coil expansion in FIG. 10 impacts an effective diameter of the primary drainage path;

FIG. 12 is a schematic view of an illustrative polymeric stent that includes a shape memory component disposed within the polymeric stent;

FIG. 13 is a schematic view of an illustrative polymeric stent that includes a number of drainage ports;

FIG. 14 is a schematic view of an illustrative polymeric stent having coiled anti-migration features;

FIG. 15 is a schematic view of two illustrative polymeric stents deployed within a bifurcated body cavity proximate a stricture within the bifurcated body cavity; and

FIG. 16 is a schematic view of an illustrative polymeric stent constrained in a delivery configuration by being disposed on a delivery device.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

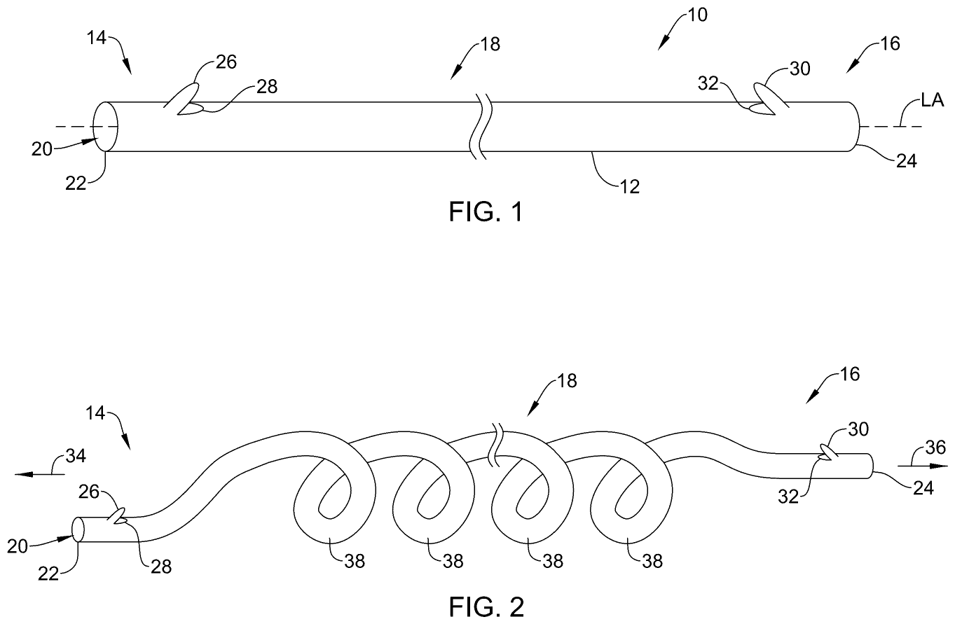

Stents are utilized in a variety of different body lumens, including the vasculature and various parts of the gastrointestinal system, for example. FIG. 1 is a schematic view of an example polymeric stent 10 that is adapted for use in body lumens in which one or more strictures have negatively impacted drainage through the body lumen. The polymeric stent 10 is shown in a delivery configuration, such as the polymeric stent 10 may have when constrained into a delivery configuration by being placed on a delivery device (not shown). The polymeric stent 10 may move into a deployed configuration that is different from its delivery configuration when the polymeric stent 10 is no longer constrained into its delivery configuration.

The polymeric stent 10 includes a polymeric tubular body 12. While the polymeric tubular body 12 is shown as generally tubular, it is contemplated that the polymeric tubular body 12 may take any cross-sectional shape desired. The polymeric stent 10 may be considered as including a first end region 14, a second end region 16 and an intervening intermediate region 18. As an example, the first end region 14 may be a distal end region and the second end region 16 may be a proximal end region, but of course these designations depend on the orientation in which the polymeric stent 10 will ultimately be implanted within a body lumen. The polymeric stent 10 may include a lumen 20 that extends from a first end 22 of the polymeric stent 10 to a second end 24 of the polymeric stent 10 to allow for the passage of food, fluids, etc. As an example, the first end 22 may be a distal end and the second end 24 may be a proximal end.

The polymeric tubular body 12 may be formed of any suitable polymeric material. In some cases, the polymeric tubular body 12 may be formed of one or more shape memory polymers. Shape memory polymers are polymers that can be heat-set in a remembered configuration, temporarily deformed from that remembered configuration, and then regain that remembered configuration when no longer constrained in the temporarily deformed configuration. In some cases, some shape memory polymers may shift from a temporary configuration to a remembered configuration as a result of environmental factors such as temperature and pH, for example.

In some cases, the polymeric stent 10 may include features such as anti-migration features that help to hold the polymeric stent 10 in place within a particular body lumen. As shown, the polymeric stent 10 includes a first barb 26 within the first end region 14. In some cases, the first barb 26 may be formed from the polymeric tubular body 12 by cutting an opening 28 within the polymeric tubular body 12 and bending the resulting flap outwardly to form the first barb 26. As shown, the polymeric stent 10 includes a second barb 30 within the second end region 16. In some cases, the second barb 30 may be formed from the polymeric tubular body 12 by cutting an opening 32 within the polymeric tubular body 12 and bending the resulting flap outwardly to form the second barb 30. While FIG. 1 only shows the first barb 26 and the second barb 30, the polymeric stent 10 may include any number of barbs. As will be discussed, the polymeric stent 10 may include other types of anti-migration features, such as coils. The polymeric stent 10 may be considered as having a longitudinal axis LA.

In some instances, the polymeric stent 10 may be deployed within a body cavity in order to provide patency to a body lumen that is otherwise at least partially narrowed or closed by one or more strictures, as well as to provide for a drainage path through the body lumen, including a drainage path through and past the one or more strictures. In some cases, the polymeric stent 10 may be adapted to move from its delivery configuration (as shown for example in FIG. 1) into a deployed configuration in which the polymeric stent 10 is able to provide both a primary drainage pathway and a secondary drainage pathway. In some cases, the deployed configuration may represent a remembered configuration given to the polymeric stent 10 via various processing steps including a heat setting step. The polymeric stent 10 may be adapted to be temporarily deformed or moved out of its remembered or deployed configuration when constrained by placement on a delivery device, and to return to its remembered or deployed configuration when no longer constrained by the delivery device.

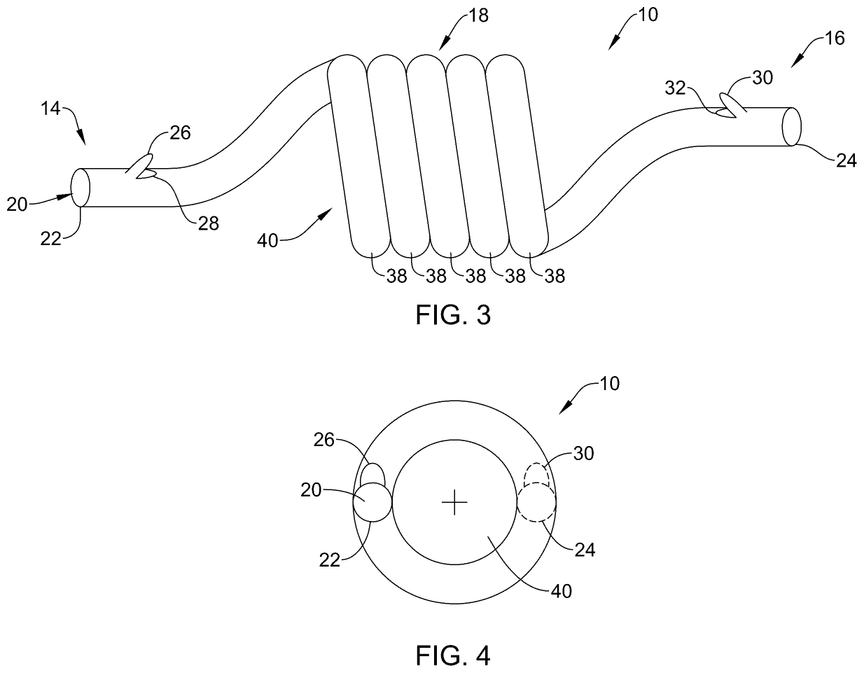

FIG. 2 is a schematic view of the illustrative polymeric stent 10 showing the polymeric stent 10 in an intermediate configuration in which the intervening intermediate region 18 has begun to move into its remembered or deployed configuration while FIG. 3 is a schematic view of the polymeric stent fully in its remembered or deployed configuration. FIG. 4 is an end view of the polymeric stent 10, taken from the left side of FIG. 3. In some cases, FIG. 2 may be considered as showing the polymeric stent 10 axially stretched from its remembered or deployed configuration, such as by pulling the first end 22 in a direction indicated by an arrow 34 and pulling the second end 24 in a direction indicated by an arrow 36 in order to better see how the intervening intermediate region 18 is coiling up. As can be seen in FIGS. 2 and 3, the intervening intermediate region 18 coils up to form a number of coil windings 38. The coil windings 38 may serve several purposes. In some cases, the coil windings 38 may exert a radial force on the body cavity in which the polymeric stent 10 is placed, causing the coil windings 38 to maintain or even improve patency of the body cavity. In some cases, the coil windings 38 may exert a radial force on any strictures that are in contact with the coil windings 38.

In some cases, the coil windings 38, because they are tightly arranged (as shown in FIG. 3), also form a drainage pathway 40 that extends through an interior volume defined by the individual coil windings 38. In some cases, “tightly arranged” may be defined as each coil windings 38 being in substantial contact with neighboring coil windings 38. This may include neighboring coil windings 38 being in physical contact over fifty percent or more, or over sixty percent or more, or over seventy percent or more, or over eighty percent or more, or over ninety percent or more of a circumferential surface of each coil winding 38.

The drainage pathway 40 may be considered as being a primary drainage pathway while the lumen 20 extending through the polymeric tubular body 12 may be considered as being a secondary drainage pathway. In some cases, the drainage pathway 40 formed by the individual coil windings 38 may have a diameter that is larger, if not substantially larger, than the secondary drainage pathway defined by the lumen 20. Because the drainage pathway 40 is larger than the secondary drainage pathway defined by the lumen 20, the drainage pathway 40 has a larger volumetric capacity and may be less likely to become clogged with solids entrained within the fluid draining through the polymeric stent 10. As an example, the lumen 20 may have an inner diameter that is in a range of about 1 millimeter (mm) (about 0.039 inches) to about 3.5 mm (about 0.14 inches), while the drainage pathway 40 may effectively have an inner diameter that is in a range of about 3 mm (about 0.12 inches) to about 10 mm (about 0.39 inches). In some cases, the polymeric stent 10 may have an overall length (in the delivery configuration) that ranges from about 25 centimeters (cm) (about 9.84 inches) to about 100 cm (about 39.4 inches).

In some cases, as shown for example in FIG. 4, the first end region 14, which includes the first barb 26, may be positioned at or near an outer periphery of the polymeric stent 10 when the polymeric stent 10 is in its remembered or deployed configuration. Similarly, the second end region 16, which includes the second barb 30, may be positioned at or near an outer periphery of the polymeric stent 10 when the polymeric stent 10 is in its remembered or deployed configuration. As a result, the first barb 26 and the second barb 30 are positioned to more likely engage tissue and help hold the polymeric stent 10 in position.

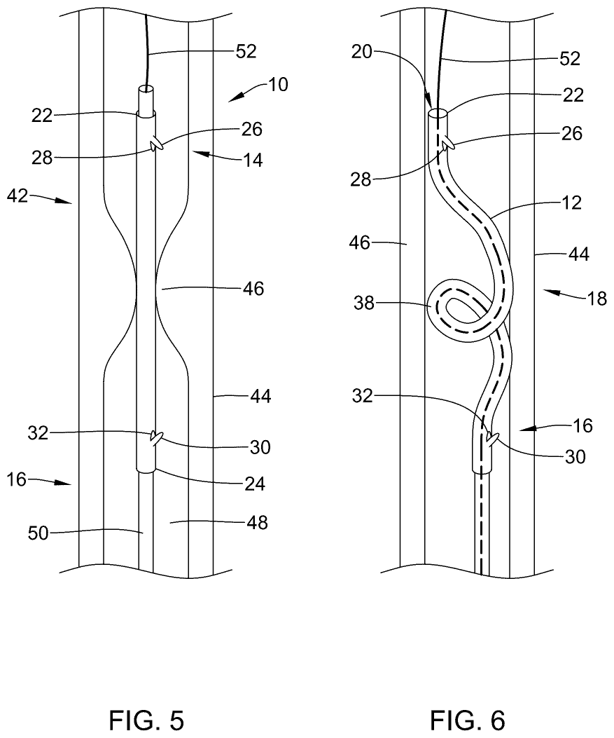

FIGS. 5, 6 and 7 are schematic views of the polymeric stent 10 in combination with a delivery device 42, showing a step by step process for deploying the polymeric stent 10 within a body cavity 44. The body cavity 44 may generally represent any of a variety of different body cavities, such as but not limited to the biliary duct and associated vessels. The body cavity 44 includes a stricture 46 that reduces an effective diameter of the body cavity 44 relative to a volume 48 outside of where the stricture 46 is located. The delivery device 42, which is adapted to extend through the polymeric stent 10, includes a delivery device shaft 50 adapted to be advanced over a guidewire 52. To begin, the guidewire 52 may be advanced into and through the body cavity 44, as shown. Next, the delivery device 42 bearing the polymeric stent 10 disposed on the delivery device shaft 50, may be advanced over the guidewire 52 in order to position the polymeric stent 10 such that the polymeric stent 10 extends through the stricture 46.

In FIG. 6, the delivery device 42 has been withdrawn proximally, which allows the polymeric stent 10 to begin transforming into its deployed configuration. As the polymeric stent 10 begins to transform into its deployed configuration, i.e., the intervening intermediate region 18 begins to coil, the coil windings as they form will exert a radially outward force, thereby flattening the stricture 46. In some cases, as shown, the guidewire 52 may remain in position, extending through the polymeric stent 10, in order to help hold the polymeric stent 10 in position relative to the stricture 46. In some cases, the guidewire 52 may be withdrawn with the delivery device 42. In some cases, the guidewire 52 may be withdrawn prior to withdrawing the delivery device 42. In some instances, the guidewire 52 may be withdrawn after withdrawing the delivery device 42. As the delivery device 42 is withdrawn, regardless of whether the guidewire 52 has been withdrawn, the polymeric stent 10 will continue to transform into its deployed configuration.

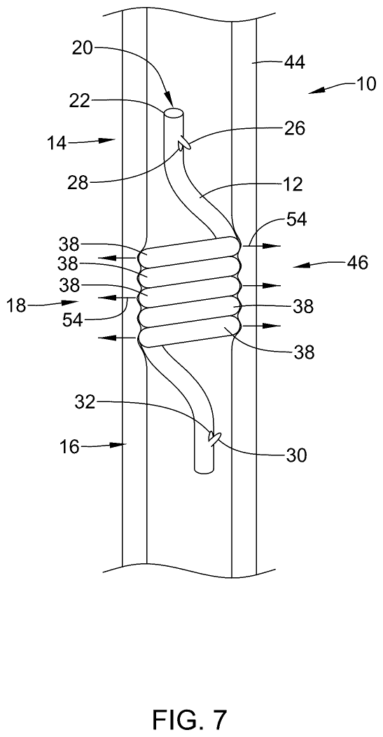

FIG. 7 shows the polymeric stent 10 in its deployed configuration within the body cavity 44. The delivery device 42 and the guidewire 52 have both been withdrawn. The intervening intermediate region 18 has transformed into a number of individual coil windings 38 that may be considered as being tightly arranged. In some cases, “tightly arranged” may be defined as each coil windings 38 being in substantial contact with neighboring coil windings 38. This may include neighboring coil windings 38 being in physical contact over fifty percent or more, or over sixty percent or more, or over seventy percent or more, or over eighty percent or more, or over ninety percent or more of a circumferential surface of each coil winding 38. As shown, the coil windings 38 may be dimensioned such that the polymeric stent 10 has an outer diameter in its deployed configuration that is larger than an inner diameter of the body cavity 44, or certainly larger than an inner diameter of the original stricture 46. As a result, the coil windings 38 may exert an outwardly radial force on the body cavity 44 and the stricture 46, as indicated by arrows 54.

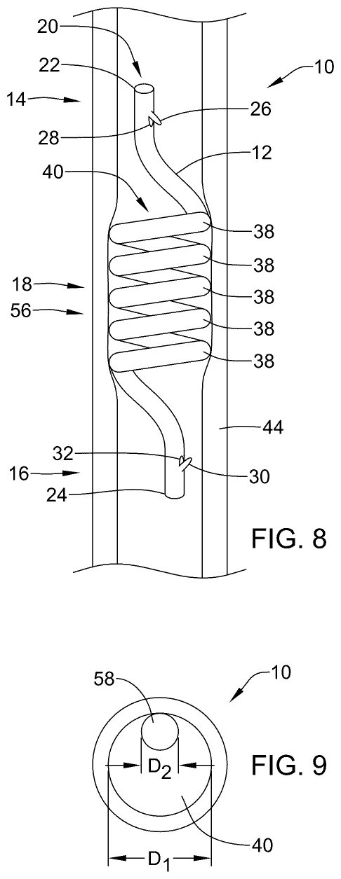

In some cases, the relative expansion of the intervening intermediate region 18, including the relative spacings between neighboring coil windings 38, and a relative angle of the coil windings 38 relative to the longitudinal axis LA (FIG. 1), may impact the overall diameter of the polymeric stent 10 in its deployed configuration. This can impact how much the stricture 46 may be pushed open. This can also impact the diameter of the drainage pathway 40 that is formed by the tightly arranged coil windings 38. FIG. 8 is a schematic view of the polymeric stent 10 disposed within the body cavity 44 showing a first level of coil expansions. Due to the relative spacing between the individual coil windings 38, or an overall diameter of each of the individual coil windings 38, or the angle relative to the longitudinal axis LA (FIG. 1) of each of the individual coil windings 38, or any combination thereof, an overall diameter of the intervening intermediate region 18 may vary. As an example, having the individual coil windings 38 arranged orthogonally to a longitudinal axis LA (FIG. 1) will result (for a given coil winding diameter) in a maximum overall diameter of the intervening intermediate region 18 will result in a maximum outer diameter of the intervening intermediate region 18, and a maximum inner diameter of the primary drainage pathway 40. Having the individual coil windings 38 arranged at an acute angle relative to a longitudinal axis LA (FIG. 1) will result (for a given coil winding diameter) in a reduced overall diameter of the intervening intermediate region 18, a reduced maximum outer diameter of the intervening intermediate region 18, and a reduced inner diameter of the primary drainage pathway 40.

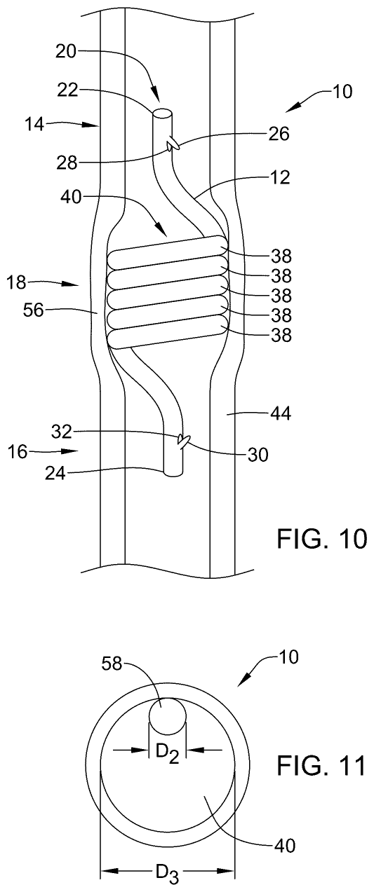

The dimensions of a radially expanded portion 56 of the body cavity 44, may vary. FIG. 9 is not a true cross-sectional view of the polymeric stent 10, but rather schematically compares a diameter D1 of the primary drainage path 40 and a diameter D2 of the secondary drainage path 58 (defined by an inner diameter of the lumen 20). FIG. 10 is a schematic view of the polymeric stent 10 disposed within the body cavity 44 showing a second level of coil expansion greater than that shown in FIG. 8. This can result in the intervening intermediate region 18 having a larger overall diameter than that shown in FIG. 8. FIG. 11 is not a true cross-sectional view of the polymeric stent 10, but rather schematically compares a diameter D3 of the primary drainage path 40 and a diameter D2 of the secondary drainage path 58 (defined by an inner diameter of the lumen 20). As can be seen, the diameter D3 of the primary drainage path 40 as shown in FIG. 11 is larger than the diameter D1 of the primary drainage path 40 as shown in FIG. 9. Similarly, the radially expanded portion 56 of the body cavity 44 has a larger diameter in FIG. 10 than it does in FIG. 8.

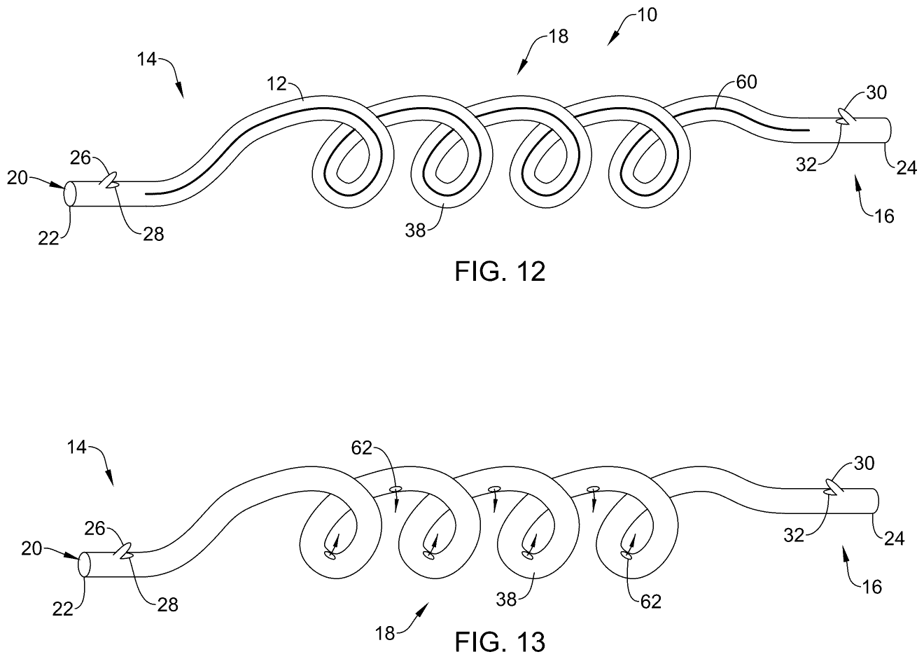

While the polymeric stent 10 may be formed of a shape memory polymer, in some cases it may be beneficial to include an additional shape memory element such as a shape memory metal wire within the polymeric tubular body 12 in order to aid the polymeric tubular body 12 in reaching its final deployed configuration. FIG. 12 shows the polymeric stent 10 as including a shape memory element 60 that is disposed within the polymeric tubular body 12. While the shape memory element 60 is shown as being disposed within (such as molded within) the polymeric tubular body 12, in some cases the shape memory element 60 may instead be exterior to the polymeric tubular body 12. As an example, the shape memory element 60 may be a Nitinol wire. Not only will the shape memory element 60 facilitate the polymeric tubular body 12 reaching its remembered or deployed configuration, but in some cases the shape memory element 60 may also provide the polymeric stent 10 with improved radiopaque characteristics that may allow better visualization of the polymeric stent 10 during post-placement evaluation processes.

FIG. 13 is a schematic view of the polymeric stent 10, showing how the polymeric tubular body 12 may include a number of drainage ports 62 that are formed within the polymeric tubular body 12 and are fluidly coupled with the lumen 20 extending through the polymeric tubular body 12. Accordingly, fluid passing through the secondary drainage pathway (such as the secondary drainage pathway 58 shown in FIGS. 9 and 11) may be allowed to exit the lumen 20 and enter the primary drainage pathway 40. In some cases, even if the lumen 20 becomes blocked at some intermediate point between the first end 22 and the second end 24, fluid may be able to continue to flow through the lumen 20 upstream of the blockage because the fluid is able to exit the lumen 20 through one or more of the drainage ports 62. The drainage ports 62 may be formed anywhere along the length of the polymeric stent 10. In some cases, at least some of the drainage ports 62 may be disposed within the intervening intermediate section 18, and as a result, at least some of the drainage ports 62 may be disposed within at least some of the coil windings 38.

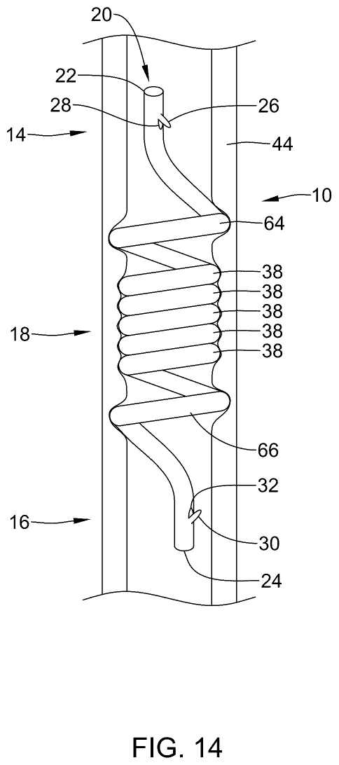

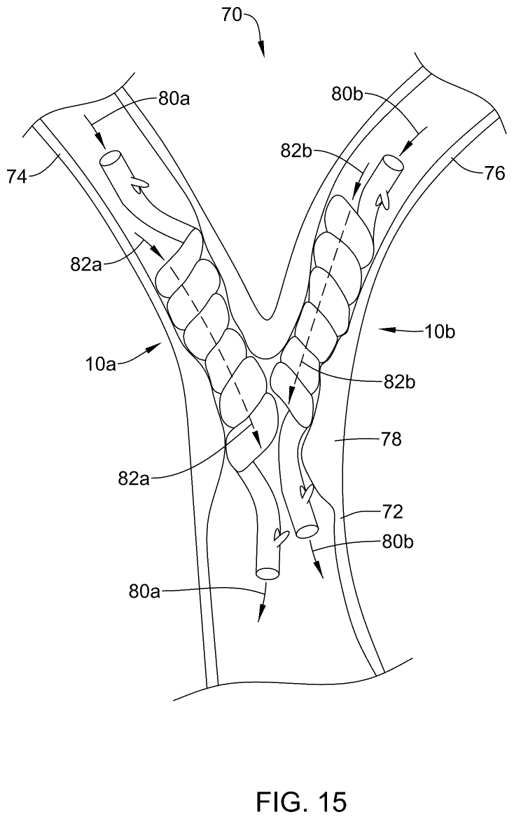

In some cases, the polymeric stent 10 may include one or more barbs that function as anti-migration elements. In some cases, the polymeric stent 10 may include one or more coils that function as anti-migration elements. FIG. 14 shows the polymeric stent 10 disposed within the body cavity 44. In addition to the intervening intermediate section 18 including a number of tightly arranged coil windings 38, the polymeric stent 10 may include one or more of a first anti-migration coil 64 and a second anti-migration coil 66. In some cases, the polymeric stent 10 may include only the first anti-migration coil 64 or only the second anti-migration coil 66, for example. In some cases, the first anti-migration coil 64 and/or the second anti-migration coil 66 have an outer diameter that is greater than an outer diameter of any of the individual coil windings 38 in order to more effectively anchor the polymeric stent 10 in place such that the polymeric stent 10 is better able to resist any applied forces.

FIG. 15 provides an example of using several polymeric stents 10 within a bifurcated body cavity 70. The bifurcated body cavity 70 includes a main portion 72, a first distal branch 74 and a second distal branch 76. A stricture 78 is shown just downstream of the bifurcation between the main portion 72, the first distal branch 74 and the second distal branch 76. A first polymeric stent 10a has been implanted within the first distal branch 74 and extends proximally into the main portion 72. A second polymeric stent 10b has been implanted within the second distal branch 76 and extends proximally into the main portion 72. Arrows 80a and 80b indicate flow through secondary drainage pathways extending through the first polymeric stent 10a and the second polymeric stent 10b, respectively. Arrows 82a and 82b indicate flow through primary drainage pathways defined by the coiled sections of the first polymeric stent 10a and the second polymeric stent 10b. Providing both primary drainage pathways and secondary drainage pathways helps to maintain patency of the body cavity 70 as well as maintaining good drainage of the body cavity 70.

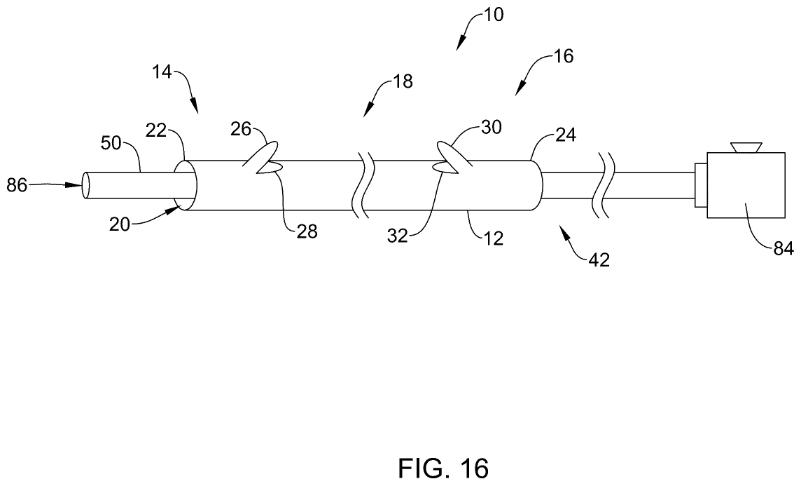

FIG. 16 provides a schematic example of the polymeric stent 10 disposed on the delivery device 42 for subsequent delivery. As shown, the polymeric stent 10 is constrained into a linear or at least substantially linear (defined as within ten percent of linear) configuration by virtue of extending over the delivery device shaft 50. The delivery device 42 includes a delivery mechanism schematically illustrated at 84. While no guidewire is shown in FIG. 16, a guidewire such as the guidewire 52 may extend through a lumen 86 extending through the delivery device shaft 50. Accordingly, once the guidewire 52 has been advanced to a particular treatment site, the delivery device 42 (and the polymeric stent 10) may be advanced over the guidewire 52 to reach the particular treatment site by accommodating the guidewire 52 within the lumen 86 extending through the delivery device shaft 50.

The materials that can be used for the various components of the medical stent(s), and the various elements thereof disclosed herein may include those commonly associated with medical devices. For simplicity purposes, the following discussion refers to the apparatus. However, this is not intended to limit the devices and methods described herein, as the discussion may be applied to other elements, members, components, or devices disclosed herein, such as, but not limited to, the medical stent and/or elements or components thereof. In some instances, the apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A polymeric stent adapted for placement within a body cavity to provide a primary drainage pathway and a secondary drainage pathway for the body cavity, the polymeric stent comprising:
a polymeric tubular body including a first end region, a second end region and an intervening intermediate region;
the polymeric tubular body having a remembered configuration in which the intervening intermediate region of the polymeric tubular body forms a plurality of tightly arranged helical coil windings that together define the primary drainage pathway;
the polymeric tubular body having a delivery configuration different from the remembered configuration when constrained by a delivery device.

2. The polymeric stent of claim 1, wherein the polymeric tubular body defines a lumen extending therethrough, the lumen defining the secondary drainage pathway.

3. The polymeric stent of claim 2, wherein the polymeric tubular body further comprises one or more drainage ports fluidly coupled with the lumen.

4. The polymeric stent of claim 1, wherein the polymeric tubular body comprises a shape memory polymer.

5. The polymeric stent of claim 1, further comprising a shape memory element secured relative to the polymeric tubular body.

6. The polymeric stent of claim 1, further comprising one or more anti-migration features disposed within at least one of the first end region and the second end region.

7. The polymeric stent of claim 6, wherein the one or more anti-migration features include a barb.

8. The polymeric stent of claim 6, wherein the one or more anti-migration features comprise a portion of the polymeric tubular body coiled into a coil winding.

9. The polymeric stent of claim 1, wherein the plurality of tightly arranged helical coil windings defining the primary drainage path are substantially orthogonal to a longitudinal axis of the polymeric stent.

10. A polymeric stent adapted for placement within a body cavity to provide drainage through the body cavity, the polymeric stent comprising:
a shape memory polymer tubular body defining a lumen extending therethrough, the shape memory polymeric tubular body having a delivery configuration when constrained by a delivery device and a deployed configuration when not constrained by a delivery device;
wherein in the deployed configuration, the shape memory polymeric tubular body forms a plurality of tightly arranged helical coil windings that together define a drainage pathway.

11. The polymeric stent of claim 10, wherein the shape memory polymeric tubular body comprises a first end region, a second end region and an intervening intermediate region that forms the plurality of tightly arranged helical coil windings defining the primary drainage pathway.

12. The polymeric stent of claim 11, wherein at least one of the first end region and the second end region includes one or more anti-migration features.

13. The polymeric stent of claim 12, wherein the one or more anti-migration features include a barb.

14. The polymeric stent of claim 12, wherein the one or more anti-migration features comprise a portion of the polymeric tubular body coiled into a coil winding.

15. The polymeric stent of claim 10, further comprising one or more drainage ports disposed within the polymeric tubular body and fluidly coupled with the lumen.

16. The polymeric stent of claim 10, further comprising a shape memory element secured relative to the polymeric tubular body.

17. A polymeric stent adapted for placement within a body cavity to provide a primary drainage pathway and a secondary drainage pathway for the body cavity, the polymeric stent comprising:
a polymeric tubular body including a first end region, a second end region and an intervening intermediate region;
one or more anti-migration features disposed within at least one of the first end region and the second end region;
the polymeric tubular body having a remembered configuration in which the intervening intermediate region of the polymeric tubular body forms a plurality of tightly arranged helical coil windings that together define the primary drainage pathway;
the polymeric tubular body having a delivery configuration different from the remembered configuration when constrained by a delivery device.

18. The polymeric stent of claim 17, wherein the one or more anti-migration features include a barb.

19. The polymeric stent of claim 17, wherein the one or more anti-migration features comprise a portion of the polymeric tubular body coiled into a coil winding.

20. The polymeric stent of claim 17, wherein the polymeric tubular body includes a lumen extending therethrough, the lumen defining the secondary drainage pathway.
